(19) United States
(12) Patent Application Publication
(10) Pub. No.: US 2021/0363673 A1
(43) Pub. Date: Nov. 25, 2021

(54) REMOVABLE PATCH WITH ACTIVE AGENT FOR TEXTILES

(71) Applicant: Textile-Based Delivery, Inc., Conover, NC (US)

(72) Inventors: David Scott Fogleman, Randleman, NC (US); Michael A. Drzewinski, Lake Hopatcong, NJ (US); Jordan Schindler, Charlotte, NC (US); Donald Ray White, JR., China Grove, NC (US)

(73) Assignee: Textile-Based Delivery, Inc., Conover, NC (US)

(21) Appl. No.: 17/326,865

(22) Filed: May 21, 2021

Related U.S. Application Data
(60) Provisional application No. 63/029,195, filed on May 22, 2020.

(51) Int. Cl. D03D 15/283; D03D 1/00

(52) U.S. Cl. CPC D03D 15/283; D03D 1/00; A61F 5/0109; D10B 2401/061; D10B 2501/04; D10B 2401/021

(57) ABSTRACT

A reversibly removable patch or section for textiles, is disclosed and described, the patch or section configured to deliver active compounds to a user, as well as textiles comprising such patches and sections. The patches and sections are configured to provide an amount of active even after repeated uses, wears, applications, and/or launderings and further provide for replacement, thus, extending the usefulness of the textile.

### CROSS-REFERENCE TO RELATED APPLICATIONS

This application claims the benefit of U.S. Provisional Application No. 63/029,195 filed on May 22, 2020, the entirety of which is incorporated herein by reference.

### TECHNICAL FIELD

The present disclosure relates to a reversibly removable patch or section for textiles, the patches or sections configured to deliver active compounds to a user, as well as textiles comprising such patches and sections. The patches and sections are configured to provide an initial amount of active even after repeated uses, wears, applications, and/or launderings and further provide for replacement, thus, extending the usefulness of the textile.

### BACKGROUND

Manufacture of textiles with the inclusion of additives is generally known. For example, FIGS. 1A and 1B show a commercially available textile, i.e., a knee brace, and in an “inside-out” configuration, respectively, the knee brace having a generally cylindrical tube shape with opposite openings 12, 14, constructed of an elastomeric yarn or fabric for formfitting constriction about the knee of a subject and further having a therapeutic active delivering section 17 positioned about a remainder section 13 of the knee brace, the therapeutic active delivering section 17 being integrated with the knee brace, for example via stitching 18. Upon depletion of the therapeutic active from therapeutic active delivering section 17, the knee brace 10 functions solely as a formfitting constrictive knee brace, without the benefit of the delivery of the therapeutic active.

### SUMMARY

One general aspect includes a patch or a section reversibly coupled to a woven or knitted fabric, the patch or the section comprising (i) a yarn; and (ii) a polymeric matrix comprising: a polymer; and an active compound dispersed in the polymer, where the polymer matrix is imbibed on the yarn.

In one aspect, alone or in combination with any one of the previous aspects, at least a portion of the woven or the knitted fabric comprises elastomeric fabric. In one aspect, alone or in combination with any one of the previous aspects, the patch or the section is elastomeric knitted fabric or elastomeric woven fabric. In one aspect, alone or in combination with any one of the previous aspects, the active compound is imbibed on the elastomeric knitted fabric or the elastomeric woven fabric. In one aspect, alone or in combination with any one of the previous aspects, the patch or the section further comprises additional yarn in a woven or knitted relationship, the additional yarn absent the active.

In one aspect, alone or in combination with any one of the previous aspects, the active compound is present in an amount for therapeutically effective delivery of the active compound to at least a portion of skin of a mammal. In one aspect, alone or in combination with any one of the previous aspects, the active compound is a medication for treating or alleviating the pathological effects or symptoms of a disease or condition. In one aspect, alone or in combination with any one of the previous aspects, the active compound is one or more compounds selected from methyl salicylate, ethyl salicylate, arnica oil, peppermint oil, menthol, lidocaine, benzocaine, capsaicin, nonivamide, niacinamide, cannabidiol, caffeine, and hydrocortisone.

In one aspect, alone or in combination with any one of the previous aspects, the polymer matrix comprises a hydrophobic polymer. In one aspect, alone or in combination with any one of the previous aspects, the polymer matrix is cross-linked. In one aspect, alone or in combination with any one of the previous aspects, the polymer matrix comprises a cross-linked hydrophobic polymer and a crystalline, semi-crystalline, or an amorphous solid active compound. In one aspect, alone or in combination with any one of the previous aspects, the textile further comprises an indicator configured to determine a qualitative or quantitative amount of wash cycles experienced by the patch or the section. of any one of the previous claims, further comprising an indicator configured determine a qualitative or quantitative amount of the active remaining in the patch or the section.

One general aspect includes an article of clothing comprising a woven or a knitted fabric; a patch or a section reversibly coupled to the woven or the knitted fabric, the patch or the section comprising a yarn, and (ii) a polymeric matrix comprising a polymer; and an active compound dispersed in the polymer, where the polymer matrix is imbibed on the yarn.

In one aspect, alone or in combination with any one of the previous aspects, the patch or the section further comprises additional yarn in a woven or knitted relationship, the additional yarn absent the active.

One general aspect includes a method for manufacturing a textile for delivery of an active compound. The method includes providing a fabric or article of clothing; and providing a patch or a section reversibly coupled to the fabric or the article of clothing or configured for reversibly coupling to the fabric or the article of clothing, the patch or the section comprising a yarn, and (ii) a polymeric matrix comprising a polymer; and an active compound dispersed in the polymer, where the polymer matrix is imbibed on the yarn.

In one aspect, alone or in combination with any one of the previous aspects, the active compound is a therapeutic active. In one aspect, alone or in combination with any one of the previous aspects, the therapeutic active is selected from the group consisting of methyl salicylate, ethyl salicylate, arnica oil, peppermint oil, menthol, lidocaine, benzocaine, capsaicin, nonivamide, niacinamide, cannabidiol, caffeine, and hydrocortisone.

One general aspect includes a reversibly attachable patch configure for coupling with a textile or article of clothing, the reversibly attachable patch comprising (i) a yarn or a fabric and (ii) a polymeric matrix comprising: a polymer; and an active compound dispersed in the polymer, wherein the polymer matrix is imbibed on the yarn or the fabric.

### BRIEF DESCRIPTION OF THE DRAWINGS

The written disclosure herein describes illustrative examples that are non-limiting and non-exhaustive. Reference is made to certain of such illustrative examples that are depicted in the figures, in which:

FIG. 1A is a front perspective view of a related active-delivering knee brace;

FIG. 1B is an inside-out perspective view of the knee brace of FIG. 1A;

FIG. 2A is a front perspective view of a textile with a reversibly removable active delivering patch or section as disclosed and described herein;

FIG. 2B is a back perspective view of The textile of FIG. 2A;

FIG. 3 is a front perspective view of an exemplary knee brace shown in an inside-out configuration with a reversibly removable active delivering patch or section as disclosed and described herein;

FIG. 4 is a side perspective view of an exemplary sleeve brace with a reversibly removable active delivery patch or section as disclosed and described herein;

FIG. 5A is a perspective view of a skin facing side of an exemplary back brace with the reversibly removable active delivering patch as disclosed and described herein;

FIG. 5B is a perspective view of the opposite side of the exemplary back brace of FIG. 5A;

FIG. 6 is an exploded view of the exemplary back brace of FIG. 5A showing the brace, and front and back sides of the reversibly removable active delivery patch, respectively, as disclosed and described herein.

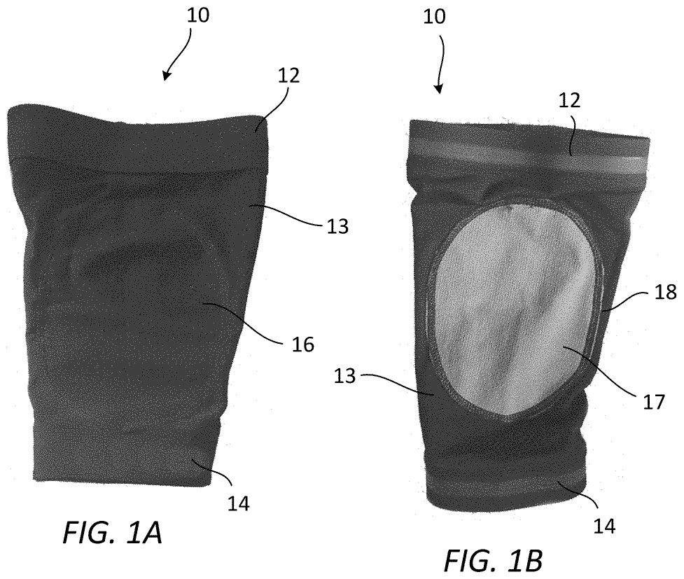

### DETAILED DESCRIPTION

The present disclosure provides reversibly removable patch or section associated with a textile, the reversibly removable patch or section comprising biologically active compounds, compositions, or ingredients (also referred to herein as “actives” and/or “therapeutic actives”) that are integrated into the constituent yarns, threads, and/or fibers, of the patch or the section. These reversibly removable patch or sections may be utilized to release the active compounds onto or into mammalian tissue, including, for example, human skin.

As used herein, the term “textile” encompasses orthopedic cast and splint materials, wound dressings, and convention tightfitting fabrics such as socks, hats, face/ski masks, scarves, tiaras, chokers, skullcaps, undergarments, skin guards, wrist bands/braces, arm bands/braces, knee pads/braces and other joint braces, bras, nylon stockings, athletic supporters, robes, neckbands, headbands, ear muffs, gloves, diapers, poultices, facial masques, pillowcases, blankets, sheets, drapes, baby strollers, and furniture coverings. Textiles can also be fabrics, woven or knitted, and foams. Textiles used herein includes commercial and non-commercial textiles or application of the presently disclose patch in commercial and non-commercial settings, such as a patch or section for public or private transportation vehicles including seats, backrests, armrests, headrests, consoles, steering wheel, or public changing tables, public stadium/entertainment seating, bar or restaurant seating, public and private waiting room seating, and other public/private settings were textiles with removable/replaceable active-releasing patches or sections that release an active over time would provide a public health benefit.

As used herein, the terms “yarn” and “yarn precursor” include not only finished yarns, but also starting or intermediate fiber-based materials from, e.g., greige cotton or extruded filament, to finished—and as described in certain examples, functionalized—yarns (e.g., yarns that are loaded with an active compound), whether on, e.g., a cone or spool or in a textile or fabric. The term “yarn” can also be used to describe individual threads and spun and/or twisted threads. In one example, the yarn may be bulked or textured. Bulked and/or textured yarns can refer to yarns that have been treated mechanically, chemically, or physically (e.g., tension-adjusted) so as to appear to have greater or increased volume relative to the yarn prior to mechanical, chemical, or physical treatment. For example, bulked and/or textured yarns can have a crimped, coiled, or spiral configuration rather than a linear or stretched configuration. Bulked and/or textured yarns can exhibit favorable properties over, e.g., partially-oriented yarn (POY) or other yarns lacking texture and/or bulk.

The present disclosure provides drug-eluting yarns, yarn precursors, threads, fibers, of the patch or sections that allow for ready integration into or use with existing commercial textile practices and materials. Highly desirable drug-delivery features such as zero order or near-zero-order release kinetics, high loading of active (e.g., drug), stabilization of active, and compatibility with various types of actives are also achieved. The examples disclosed herein may be used for improving the health of skin via local delivery of dermatological actives, but are also capable of transdermal delivery of skin-permeable actives and numerous other applications, as described in greater detail below.

In one example, yarns comprising extruded fibers are used. For example, synthetic yarns (e.g., nylon, polyester, etc.) can include extruded fibers. In one example, the patch or section is a fabric comprising a nylon, polyester or acrylic material that requires elasticity or stretch. Thus, the yarn may be plied or twisted with an air-covered yarn (e.g., spandex) to enable additional stretch of the yarn. Additionally, the yarn may be air-covered/air-intermingled (i.e., blowing air onto the yarn and adding a spandex core into the middle of the yarn). These methods are particularly useful for garments that need a lot of stretch such as tights or leggings (or even the elastic portion on the tops of socks).

In one example, the present disclosure provide patches or sections that release at a constant or near-constant rate over most of the duration of an extended release profile. As discussed above, the examples of this disclosure can be configured to achieve a constant or near-constant rate of release of an active compound from the patch or the section. This is of particular value for an active that has a relatively low therapeutic index such that systemic levels should be kept as constant as possible over time, or when a diffusion-limited t^{1/2} profile would waste much of the active during an early-time high release rate.

In one example, patches and/or sections that release at a near-constant rate per area of skin contact over a dominant portion of the duration of an extended release profile provide constancy of release of the active.

Imbibition, as that term is used herein, refers to the act of contacting a filament, fiber, thread, yarn, or woven or knitted textile with a polymer matrix, and results in the taking up the polymer matrix or a precursor thereof (e.g., monomer or a pre- or partially cured polymer matrix) to a significant or threshold imbibition loading, for example, between about 5% and about 1,000% of the substrate weight, between about 10% and about 200% of the substrate weight, or between about 20% and about 100% of the substrate weight. The substrate may be, for example, yarn, yarn precursor, thread, fiber, or some other component of the patch or section.

As used herein, “Yarn-level imbibition” refers to individual yarns that are imbibed. As used herein, “Warp imbibition” refers to yarns or fibers, of weaving process, where the “warp” yarns or fibers are imbibed. Likewise, as used herein, “Weft imbibition refers to yarns or fibers, of weaving process, where the “weft” yarns or fibers are imbibed. As used herein, “Textile-level imbibition” refers to a two-dimensional textile or fabric that is imbibed.

In certain examples, the reversibly releasable patch or section comprises the both medicated or active releasing yarn of this disclosure along with ordinary, non-active containing yarn (additional yarn). For example, in woven textiles, the warp can be traditional yarn and the weft yarn comprising active. In other examples, only medicated yarn may be used. Thus, in one example, the patch or the section comprises an additional yarn in a woven or knitted relationship with imbibed yarn, the additional yarn being absent the active.

According to the present disclosure, large dosages of several grams or more per dosing, that are difficult to deliver as pills or in other dosing forms, can be administered through skin-contacting patches or sections of clothing in a way that is convenient, private, and even fashionable, for example, by lying on a pillow at night, or putting on socks or another article of clothing so as to provide contact with the medicated material.

Certain areas of the body may be well-suited to delivery of an active substance via a patch or section rather than the entire article of clothing. For example, portions of the lower back, thighs, knees, elbows etc. are particularly well-suited to targeted active delivery rather than administration throughout the entire article of clothing. Further, the patch and/or section as presently disclosed can be replaced, for example, with a different dosing regimen and/or different therapeutic active medicated and be removable for washing of the article of clothing independently of the patch or section.

Specific classes of compounds that can be incorporated as actives and delivered via the presently disclosed patch or section include demulcents, emollients, lubricants, vasoconstrictors, antibiotics and antiseptics, antihistamines, immunosuppressants, local anesthetics, antiallergics, antifungals, vasoprotectants, anticoagulants, mucolytic and proteolytic compounds, antiglaucoma drugs, and anti-inflammatories, anesthetics, anti-helminthic, analgesics, steroids, non-steroidal inhibitors of the inflammatory cascade, anti-neoplastic, anti-angiogenic, calcineurin inhibitors, anti-ocular hypertensives, antivirals, antibacterials, neuroprotectants, anti-apoptotics, medications for dry eye, pupil dilating medications (mydriatics and cycloplegics), ocular decongestants, antioxidants, photosensitizers, photodynamic therapy agents, mast cell stabilizers, monoclonal antibodies, quinolone antibiotics, and intra-ocular pressure lowering agents. Also suitable are derivatives, analogs, and prodrugs, and mixtures and combinations thereof.

In certain examples, actives that are dyed or colored may be utilized. Colored actives provide several potential advantages, such as providing the user visual confirmation of activity, favorably modifying skin color or tone, and aiding in manufacturing QA/QC. Colored actives that may be incorporated into yarns of the examples include, but are not limited to, Curcumin, Methylene Blue, Gentian Violet, Dantrolene sodium, and Oil Red O. These actives cover a range of therapeutic effects including anticancer, antibacterial, antifungal, antispasmotic, antioxidant and anti-inflammatory effects. In other examples, an active indicator can be used, such as disclosed in co-assigned, co-pending application No. 63/029,175 filed May 22, 2020, the disclosure of which is incorporated herein by reference. In other examples, a wash cycle indicator can be used, such as disclosed in co-assigned, co-pending application No. 63/029,125 filed May 22, 2020, the disclosure of which is incorporated herein by reference.

For application of actives to portions of skin suffering from abnormalities or for cosmetic improvement, the presently disclosed reversibly releasable patches or sections offer direct skin contact, localizable coverage, washing machine compatibility (“washability”), rapid rate of release, continuous coverage through the night if desired or throughout the day or night. In one example, the reversibly releasable patch or section of the present disclosure is used as an insert or lining to a cast, splint, sling or brace. The cast/insert system, in one example, is designed such that the insert is reversibly removable, daily if necessary, for washing without interfering with the supportive and protective functions of the or for washing the brace separate and apart from the patch or section. The patch or section could further provide release of antimicrobials, growth factors, analgesics, and skin toning/cosmeceutical actives, and release medicaments or essential oils designed to increase blood circulation.

In one example, the disclosed reversibly releasable patch or section is adaptable for garments that provide a substantial amount of stretch, e.g., such as tights or leggings (or even the elastic portion at the top of a sock or undergarment) as the patch or section can be fabricated with similar yarn or fabric or a different yarn or fabric.

Semi-finished garments and articles of wear configured to reversibly receive the patch or section as well as the patch or section itself can be further treated through methods of finishing, treating, washing, dyeing, etc. the presently disclosed patch or section can be configured to be pre-washed to either remove excess dye or to soften the surface or appearance, provided with coatings for abrasion resistance, be able to withstand sharp angles and points of frictional contact so as to avoid cracking or flaking, be able to withstand processing temperatures, and have similar flexural properties to that of the fiber or yarn of the textile for which it is associated with and to move in concert with the fiber or yarn during deformation (e.g., stretching or folding).

Patches and/or sections suitable for garments and textiles containing active fibers can be tested in various ways for efficacy, e.g., measurement of active release rate and/or resistance to active loss by use, wear, application, and/or laundering. For example, a solvent extraction method on a basic, coated yarn or final garment can be conducted. The extract arising from such extractions can then be characterized, e.g., by HPLC to determine the quantity of active released based on the time and conditions of extraction. As to studying resistance to laundering, fibers, yarns, and garments can be washed under controlled conditions and time (e.g., cycles) followed by an extraction as discussed above. Comparison of extraction data before and after laundering can be used to determine the qualitative or quantitative amount of active lost.

### Polymer Matrix

The patches or sections of the present disclosure can include or utilize cross-linked, polymers as polymeric matrices for actives. Cross-linking (also referred to as “curing,” “vulcanizing,” and “thermosetting”) applied to a dispersion or suspension of active particles in a polymer, oligomer, or monomer matrix—such as a Room Temperature Vulcanizer (RTV), commercial coating or adhesive, chemically reactive linear polymer, etc.—can be employed by the various examples of the present disclosure for preparing patches and/or sections that can protect the active against excessive loss during laundering, as well as against a wide range of chemical degradation reactions including hydrolysis, oxidation (depending on the polymer), acid/base-catalyzed reactions, etc. The polymer matrices can be formed from various polymer- or oligomer-based systems, including commercially available elastomeric adhesives, glues, coatings, caulks, sealants, casting materials, and cross-linking systems. The polymers (e.g., elastomers) can also be formed from one or more monomers.

In specific examples, the polymers (e.g., elastomers) may be used as a vehicle to load one or more actives into and/or onto the yarn, yarn precursor, thread, fiber, of the patch or section and/or immobilize the one or more actives in and/or on the yarn, yarn precursor, thread, fiber, of the patch or section. For example, in particular examples, one or more actives is combined with a polymer (e.g., elastomer) to form a mixture or solution, which is imbibed by a yarn, yarn precursor, thread, fiber, of the patch or section. Some of the examples disclosed herein include cross-linking so as to lock, hold, or otherwise temporarily retain an active and protect it from degradation and premature loss, particularly in the face of stress conditions such as those encountered in laundering.

Various materials can be used to prepare the polymeric matrix of the reversibly releasable patch or section. For example, the matrix may include a polymer or an elastomer that exhibits relatively low toxicity, low allergenic potential, and/or low skin irritation. The matrix also release the active at a rate that delivers an efficacious and reasonably safe dose in the time anticipated or desired for the patch or section tissue-contact.

The selection of monomers in a curable system or the selection of a pre-made polymer coating can be varied to enhance necessary functions including, but not limited to: coupling to the yarn, fiber, etc.; compatibility with and/or the release profile of the active; physical and/or mechanical properties to aid garment creation (e.g., knitting); and/or to manage the tactile and other aesthetics of the yarns, fibers of the patch or the section.

In one example, a monofunctional monomer may be used. For example, an alkyl methacrylate or alkyl acrylate having an alkyl group having between one and 12 carbon atoms may be used. Other suitable monofunctional monomers are also within the scope of this disclosure. In certain examples an aromatic acrylate or an aromatic methacrylate may be used. For example, the aromatic acrylate or methacrylate may be selected from at least one of phenyl acrylate/methacrylate, phenylethyl acrylate/methacrylate, and/or phenoxyethyl acrylate/methacrylate. Other suitable aromatic acrylates or methacrylates are also within the scope of this disclosure.

In one example, a hydroxy-containing acrylate or a hydroxy-containing methacrylate may be used. The hydroxy-containing acrylate or methacrylate may be selected from at least one of a hydroxyalkyl acrylate/methacrylate (e.g., 2-hydroxyethyl acrylate/methacrylate), 2-hydroxypropyl acrylate/methacrylate, 4-hydroxybutyl acrylate/methacrylate, 6-hydroxyhexyl acrylate/methacrylate, and/or 8-hydroxyoctyl acrylate/methacrylate. Other suitable hydroxy-containing acrylates or methacrylates are also within the scope of this disclosure.

In certain examples, cross-linkable, multi-functional acrylates and methacrylates may be used. For example, a difunctional monomer is used. Other cross-linkable, multi-functional acrylates and methacrylates are also within the scope of this disclosure. In one example, a monomer or an oligomer may be used. In another example, the monomer or oligomer is selected from at least one of, aliphatic silicone acrylate, LED cure promoting oligomer, urethane dimethacrylate, and/or any other suitable monomer or oligomer.

Suitable oligomers include, but are not limited to, epoxy acrylates, urethane acrylates, carboxylic acid half esters, polyester acrylates, acrylated acrylics, and/or low viscosity oligomers. In one example, a polyurethane acrylate system may be used. Other suitable polyurethane acrylate systems are also within the scope of this disclosure.

The various examples of the present disclosure can include or utilize polymers, prepared in situ on the yarn, fiber of the patch or the section, coated and/or imbibed from solution, thus forming a functional coating, which serves as a protective matrix for beneficial actives and in which, in turn, controls their delivery for a releasably coupled patch or section. In certain examples, the polymer may be hydrophobic and/or cross-linked. Cross-linking (also referred to as “curing,” “vulcanizing,” and “thermosetting”) applied to a dispersion or suspension of active in a polymer, oligomer, or monomer matrix, commercial coating or adhesive, chemically reactive linear polymer, etc.—can be employed by the various examples of the present disclosure for preparing the patch or section that can protect the beneficial active against excessive loss during laundering, as well as against a wide range of chemical degradation reactions including hydrolysis, oxidation, acid/base-catalyzed reactions, etc. The various examples of the present disclosure can also be formed from one or more monomers and or polymers in any combination.

In one example, related to wound dressing, the patch or section of the present disclosure may be used as an insert or lining to a cast, splint, sling or brace. In one example, the disclosed insert or lining is employed to a cast, splint, sling, or brace. The cast/insert system could be designed such that the insert could be removed, daily if necessary, for washing without interfering with the supportive and protective functions of the cast or brace. The insert could provide release of antimicrobials, growth factors, analgesics, and skin toning/cosmeceutical actives, and release medicaments or essential oils designed to increase blood circulation.

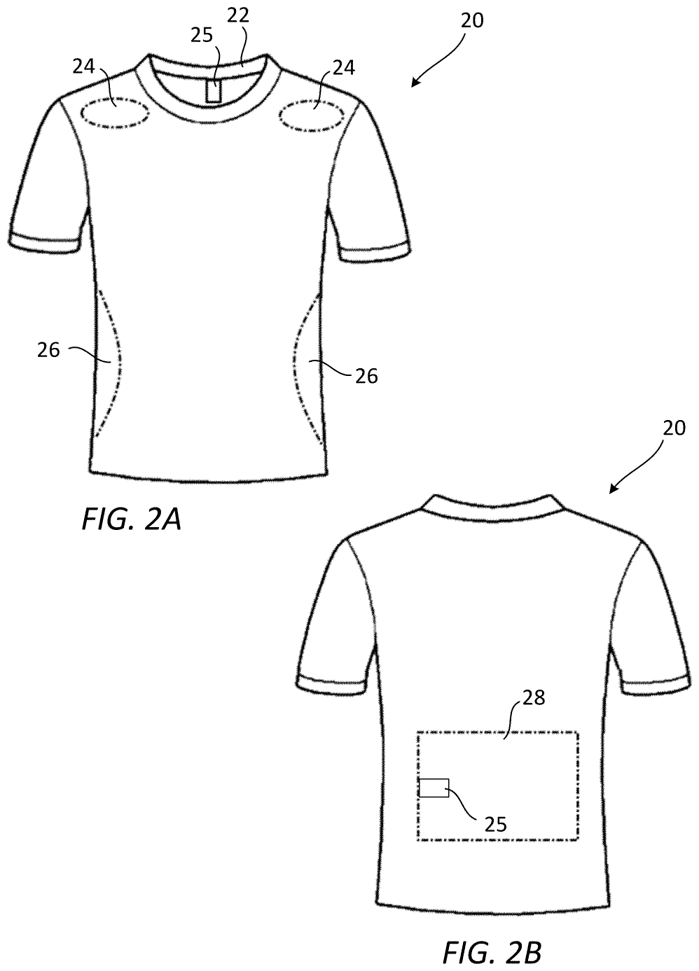

Thus, with reference to FIG. 2A a front perspective view of a textile garment 20 shown with a reversibly removable active delivering patch or section 24, 26 disposed on the front or side of the textile garment 20, as well as patch or section 28 disposed on the reverse side of garment 20 as shown in FIG. 2B. Patch or section 24,26 can be held in place by a variety of reversibly removable means such as cohesive means and/or adhesive means, hook and loops means (Velcro), fastener means such as buttons, tabs and snaps. Cohesive means can include portions or layers of materials that provide a mating surface between the patch or section and the article of clothing that resists displacement during use but otherwise can be separated by user. Such materials include polyvinyl chloride, silicones, and other low-tack materials typically used as release liners for adhesives. Adhesive means can include pressure sensitive adhesives.

In one example, the textile 20 of FIG. 2A further includes an indicator 25 positioned for example about the collar 22 or integral with the reversibly removable patch 28. In one example, indicator 25 qualitatively or quantitatively provides a visual indication of an amount of active remaining in the patch or section and/or the number of wash cycles experienced by the patch or section.

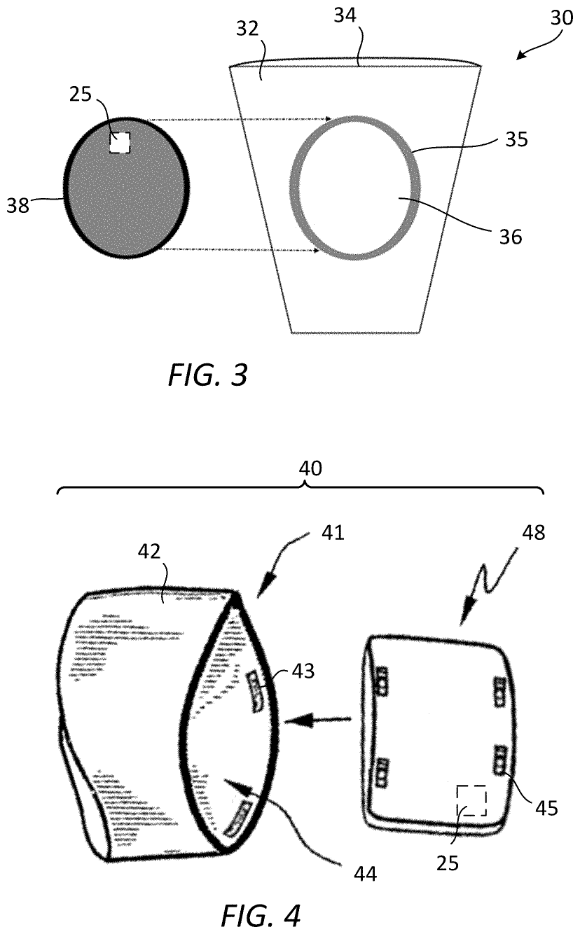

FIG. 3 is a front perspective view of an exemplary knee brace 30 (shown “inside out”) having a generally tubular shape with larger opening 34 for receiving the leg of a user, the brace 30 having a reversibly removable active delivering patch or section 38 configured for cohesive attachment to corresponding location 35 of skin facing surface 32 of brace 30. Location 35 comprises cohesive surface 36 configured to secure patch or section 38 during use and to allow for reversible removal and/or replacement during use. As shown in FIG. 3 optional indicator 25 can be provided on patch or section 38 as described above.

FIG. 4 is a side perspective view of an exemplary sleeve brace assembly 40 having sleeve with outer surface 42 configured to receive reversibly releasable active delivery patch or section 48 into opening 44. Patch or section 48 is shown as a generally tubular configuration having attachment members 45 configured for attaching to corresponding attachment members 43 of sleeve 41. As shown in FIG. 4, optional indicator 25 can be provided on patch or section 48 as described above.

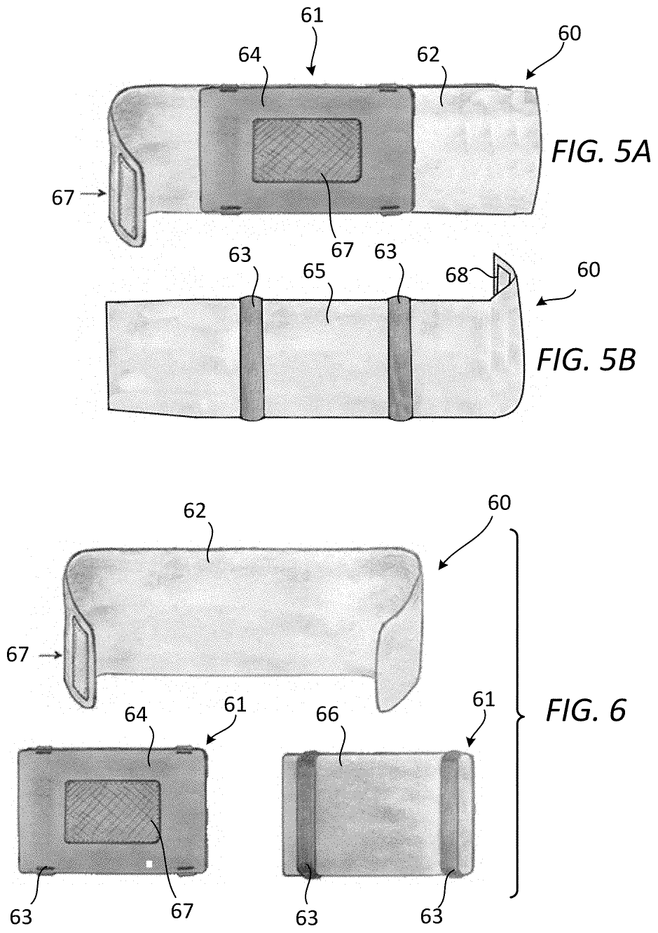

FIGS. 5A and 5B are perspective views of a skin facing side 62 and an opposing side 65 of an exemplary back brace 60 with the reversibly removable active delivering patch 61 position about back brace 60 with strap members 63. Back brace 60 includes securement member 68 at longitudinal distal end of the brace for securely wrapping brace about the lower back, for example. FIG. 6 is an exploded view of the exemplary back brace 60 showing the brace 60, and front side 64 and back side 66 of the reversibly removable active delivering patch 61 having strap member 63 positioned at opposing longitudinal ends of patch 61. Strap members 63 provide for positioning of patch 61 about brace 60.

As shown in FIG. 6, patch 61 has skin facing surface 64 with active delivery pad 67 configured to face the skin of the wearer of the brace 60. In one example, active delivery pad 67 is reversibly removable from patch 61 using the aforementioned cohesive and/or adhesive means of securement. In another example, active delivery pad 67 is integral with patch 61. In one example, either longitudinal end of brace 60 is inserted between strap member 63 so that patch 61 and can be positioned along the longitudinal length of brace 60 as desired. Thus, patch 61 functions as a sleeve that would slide on and from the back brace 60 allowing for easy on off without significantly altering the construction or function of the brace itself and how it was initially designed.

In one example, pad 67 coupled to a piece of neoprene fabric for providing stability, and allowing allow the head to press against the back of the user, and also providing additional warmth to the back area. In one example, the patch 61 is the same width as the brace, and configured of material on the back side thereof that matches the color of the material of the brace 60 so as to not look out of place. In another example, patch 61 provides compression to keep maintain correct position. The brace 60 of FIG. 6 provides for removal and wash of the patch 61, including pad 67, without having to also wash the brace.

Although the above disclosure has been presented in the context of exemplary embodiments, it is to be understood that modifications and variations may be utilized without departing from the spirit and scope of the invention, as those skilled in the art will readily understand. Such modifications and variations are considered to be within the purview and scope of the appended claims and their equivalents.

We claim:

1. A textile comprising:
a woven or a knitted fabric;
a patch or a section reversibly coupled to the woven or knitted fabric, the patch or the section comprising:
(i) a yarn; and
(ii) a polymeric matrix comprising: a polymer; and an active compound dispersed in the polymer, wherein the polymer matrix is imbibed on the yarn.

2. The textile of claim 1, wherein at least a portion of the woven or the knitted fabric comprises elastomeric fabric.

3. The textile of claim 1, wherein the patch or the section is elastomeric knitted fabric or elastomeric woven fabric.

4. The textile of claim 3, wherein the active compound is imbibed on the elastomeric knitted fabric or the elastomeric woven fabric.

5. The textile of claim 1, wherein the patch or the section further comprises additional yarn in a woven or knitted relationship, the additional yarn absent the active compound.

6. The textile of claim 1, wherein the active compound is present in an amount for therapeutically effective delivery of the active compound to at least a portion of skin of a mammal.

7. The textile of claim 1, wherein the active compound is a medication for treating or alleviating pathological effects or symptoms of a disease or condition.

8. The textile of claim 1, wherein the polymer matrix comprises a hydrophobic polymer.

9. The textile of claim 1, wherein the polymer matrix is cross-linked hydrophobic polymer.

10. The textile of claim 1, further comprising an indicator configured to determine a qualitative or quantitative amount of wash cycles experienced by the patch or the section or a qualitative or quantitative amount of the active compound remaining in the patch or the section.

11. A method for manufacturing a textile for delivery of an active compound, the method comprising:
providing a fabric or article of clothing; and
providing a patch or a section reversibly coupled to the fabric or the article of clothing or configured for reversibly coupling to the fabric or the article of clothing, the patch or the section comprising:
(i) a yarn; and
(ii) a polymeric matrix comprising: a polymer; and an active compound dispersed in the polymer, wherein the polymer matrix is imbibed on the yarn;
wherein the patch or section comprises additional yarn in a woven or knitted relationship, the additional yarn absent the active compound.

12. A reversibly attachable patch configured for coupling with a textile or article of clothing, the reversibly attachable patch comprising:
(i) a yarn or a fabric; and
(ii) a polymeric matrix comprising: a polymer; and an active compound dispersed in the polymer, wherein the polymer matrix is imbibed on the yarn or the fabric.

13. The reversibly attachable patch of claim 12, wherein at least a portion of the fabric comprises elastomeric fabric.

14. The reversibly attachable patch of claim 12, wherein the reversibly attachable patch comprises elastomeric knitted fabric or elastomeric woven fabric.

15. The reversibly attachable patch of claim 12, wherein the reversibly attachable patch further comprises additional yarn in a woven or knitted relationship with the fabric, the additional yarn absent the active compound.

16. The reversibly attachable patch of claim 12, wherein the polymer matrix comprises a hydrophobic polymer.

17. The reversibly attachable patch of claim 12, wherein the polymer matrix is cross-linked hydrophobic polymer.

18. The reversibly attachable patch of claim 12, further comprising an indicator configured to determine a qualitative or quantitative amount of wash cycles experienced by the wherein the reversibly attachable patch or a qualitative or quantitative amount of the active compound remaining in the patch.

19. An article of clothing comprising:
a woven or a knitted fabric;
a patch or a section reversibly coupled to the woven or knitted fabric, the patch or the section comprising:
(ii) a yarn; and
(ii) a polymeric matrix comprising: a polymer; and an active compound dispersed in the polymer, wherein the polymer matrix is imbibed on the yarn.

20. The article of clothing of claim 19, wherein the patch or the section further comprises additional yarn in a woven or knitted relationship, the additional yarn absent the active compound.
